(19) United States
(12) Patent Application Publication
(10) Pub. No.: US 2007/0021325 A1
(43) Pub. Date: Jan. 25, 2007

(54) Drug formulation containing a solubilizer for enhancing solubility, absorption, and permeability

(75) Inventors: Youngro Byun, Seoul (KR); Sang Kyoon Kim, Seoul (KR)

Correspondence Address: ALAN J. HOWARTH, P.O. BOX 1909, SANDY, UT, 84091-1909, US

(73) Assignee: Mediplex Corporation

(21) Appl. No.: 11/188,217

(22) Filed: Jul. 21, 2005

(51) Int. Cl. A61K 38/28; C08B 37/10; A61K 31/727; A61K 38/23

(52) U.S. Cl. 514/3; 514/12; 514/56; 530/303; 536/21

(57) ABSTRACT

Solubility, absorption, and permeability of drugs upon oral administration are improved when the drugs are mixed and/or complexed with water-miscible organic solvents. Illustratively, the absorption of a heparin-deoxycholic acid conjugate upon oral administration is increased by mixing and/or complexing this conjugate with dimethyl sulfoxide. Other illustrative water-miscible organic solvents include N-methylpyrrolidone, polyoxyl 35 castor oil, diethylene glycol monoethyl ether, and benzoic acid.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

This invention relates to drug formulations containing solubilizers for increasing solubility, absorption, and permeability of the drug. More particularly, this invention relates to mixtures and complexes of a drug and a solubilizer, wherein the solubilizer increases the water solubility of the drug and the absorption and permeability of the drug through the intestinal mucosa into the bloodstream.

Many drugs are not available in oral formulations because of poor or insufficient solubility, absorption through the intestinal mucosa, and/or permeability of the drug. Since oral formulations are highly desirable for many reasons relating to convenience and cost, it would be advantageous to develop drug formulations that exhibit increased solubility, absorption, and permeability.

### BRIEF SUMMARY OF THE INVENTION

It is a feature of the present invention to provide drug formulations comprising a mixture, a complex, or a combination of a mixture and a complex of a drug and a solubilizer, wherein the solubility, absorption, and permeability of the drug are increased upon oral administration.

An illustrative embodiment of the present invention comprises a composition comprising an aqueous mixture, complex, or combination of a mixture and a complex of a drug and a water-miscible organic solvent. Illustrative examples of such drugs include polysaccharides, proteins, polysaccharide conjugates, protein conjugates, and protein complexes, and mixtures thereof. An illustrative example of a polysaccharide drug comprises heparin, and illustrative examples of protein drugs comprise insulin and calcitonin. Similarly, heparin-bile acid conjugates and insulin- or calcitonin-bile acid conjugates are illustrative examples of polysaccharide conjugates and protein conjugates, respectively. Illustrative examples of water-miscible organic solvents according to the present invention include dimethyl sulfoxide, N-methylpyrrolidone, polyoxyl 35 castor oil, diethylene glycol monoethyl ether, benzoic acid, and mixtures thereof.

Another illustrative embodiment of the invention comprises a composition formed by a process comprising:

(a) mixing a drug and a water-miscible organic solvent in an aqueous solution to form a mixture; and

(b) evaporating the mixture to dryness.

Still another illustrative embodiment of the present invention comprises a method of treating a condition in a warm-blooded animal in need of treatment therefor, the method comprising orally administered a composition comprising a mixture, a complex, or a combination of a mixture and a complex of a drug and a water-miscible organic solvent. The composition can comprise an aqueous solution or a dried form.

Yet another illustrative embodiment of the invention comprises a composition comprising a mixture, a complex, or a combination of a mixture and a complex of heparin and dimethyl sulfoxide.

A still further illustrative example of the present invention comprises a method of treating a patient in need of anticoagulation therapy, the method comprising orally administering a safe and effective amount of a composition comprising a mixture, a complex, or a combination of a mixture and a complex of a heparin-bile acid conjugate and dimethyl sulfoxide. Heparin-deoxycholic acid conjugate is an illustrative example of such a heparin-bile acid conjugate.

Further yet, an illustrative embodiment of the present invention comprises a method of making an oral formulation for anticoagulation therapy, the method comprising:

(a) conjugating heparin to a bile acid to form a heparin-bile acid conjugate; and

(b) mixing the heparin-bile acid conjugate with an aqueous solution of a water-miscible organic solvent to form an aqueous formulation.

This method can optionally further comprise evaporating the aqueous formulation to dryness and encapsulating or tableting the dried aqueous formulation. Alternatively, the method can further comprise encapsulating the aqueous formulation.

### BRIEF DESCRIPTION OF THE SEVERAL VIEWS OF THE DRAWINGS

FIG. 1 shows FT-IR analysis of DMSO bound to a heparin-DOCA conjugate: heparin (solid line); DMSO-bound heparin-DOCA (10%; dotted line); DMSO-bound heparin-DOCA (30%, dashed line).

FIG. 2 shows a TGA profile of DMSO-bound heparin-DOCA: heparin (solid line); DMSO-bound heparin (long-dashed line); heparin-DOCA (short-dashed line); DMSO-bound heparin-DOCA (dotted line).

FIG. 3 shows a DSC profile of DMSO-bound heparin-DOCA: heparin (solid line); DMSO-bound heparin (long-dashed line); heparin-DOCA (short-dashed line); DMSO-bound heparin-DOCA (dotted line).

FIG. 4 shows oral absorption of DMSO-bound heparin-DOCA in mice: heparin-DOCA in 10% DMSO formulation (◯); DMSO-bound heparin-DOCA (□); DMSO-bound heparin (Δ).

FIG. 5 shows absorption of DMSO-bound heparin-DOCA in capsule in monkeys when taken orally: 100 mg/kg heparin in buffer (◯); 5 mg/kg DMSO-bound heparin-DOCA in capsule (□); 10 mg/kg DMSO-bound heparin-DOCA in capsule (Δ).

FIG. 6 shows an absorption profile of DMSO-bound heparin-DOCA in monkeys when taken orally: heparin in buffer (100 mg/kg; ◯); heparin-DOCA in buffer (10 mg/kg; □); DMSO-bound heparin in buffer (10 mg/kg; Δ); DMSO-bound heparin-DOCA in buffer (5 mg/kg; ∇); DMSO-bound heparin in buffer (5 mg/kg; ⋄).

### DETAILED DESCRIPTION

Before the present drug formulations for increasing solubility, absorption, and permeability of the drug are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a drug formulation containing “a drug” includes a mixture of two or more drugs, reference to “a solubilizer” includes reference to one or more of such solubilizers, and reference to “a bile acid” includes reference to a mixture of two or more bile acids.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, “DMSO” means dimethyl sulfoxide, and “NMP” means N-methyl pyrrolidone.

As used herein, “polyoxyl 35 castor oil” is a nonionic solubilizer and emulsifier produced by causing ethylene oxide to react with pharmaceutical grade castor oil in a molar ratio of 35:1. The main component of polyoxyl 35 castor oil is glycerol-polyethylene glycol ricinoleate, which, together with fatty acid esters of polyethyleneglycol, represents the hydrophobic part of the product. The smaller, hydrophilic part comprises polyethylene glycols and ethoxylated glycerol. Polyoxyl 35 castor oil has a hydrophilic-lipophilic balance (HBL) between 12 and 14.

As used herein, “DOCA” means deoxycholic acid, and “heparin-DOCA” means a conjugate of heparin and deoxycholic acid.

As used herein, “protein” means peptides of any length and includes polypeptides and oligopeptides. In other words, “protein” is used herein without any particular intended size limitation, unless a particular size is otherwise stated. Typical of proteins that can be used in the present invention are those selected from group consisting of oxytocin, vasopressin, adrenocorticotrophic hormone, epidermal growth factor, prolactin, luliberin or luteinising hormone releasing hormone, growth hormone, growth hormone releasing factor, insulin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, monoclonal antibodies and soluble vaccines. The only limitation to the protein or peptide drug that may be used in the present invention is functionality.

As used herein, “polysaccharide” means a carbohydrate containing more than three monosaccharide units per molecule, the units being attached to each other by glycoside linkages. Illustrative polysaccharides include heparin, heparin sodium, sulfonated polysaccharides, cellulose, hydroxymethylcellulose, and hydroxypropylcellulose.

As used herein, “bile acids” means natural and synthetic derivatives of the steroid, cholanic acid, including, without limitation, cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, hyodeoxycholic acid, and mixtures thereof, and the like.

As used herein, “sterols” means alcohols structurally related to the steroids including, without limitation, cholestanol, coprostanol, cholesterol, epicholesterol, ergosterol, ergocalciferol, and mixtures thereof, and the like.

As used herein, “alkanoic acids” means saturated fatty acids of about 4 to 20 carbon atoms. Illustrative alkanoic acids include, without limitation, butyric acid, valeric acid, caproic acid, caprylic acid, capric acid, lauric acid, myristic acid, palmitic acid, stearic acid, and mixtures thereof, and the like. As used herein, “effective amount” means an amount of a pharmacologically active agent that is nontoxic but sufficient to provide the desired local or systemic effect and performance at a reasonable benefit/risk ratio attending any medical treatment. Thus, for example, an effective amount of a heparin-DOCA conjugate is an amount sufficient to provide a selected level of anticoagulation activity.

As used herein, the term “safe and effective amount” refers to the quantity of a component which is sufficient to yield a desired response without undue adverse effects (such as toxicity, irritation, or allergic response) commensurate with a reasonable benefit/risk ratio when used in the manner of this invention. The specific “safe and effective amount” will, obviously, vary with such factors as the particular condition that is being treated, the severity of the condition, the duration of the treatment, the physical condition of the patient, the nature of concurrent therapy (if any), and the specific formulation used in the present invention.

The instant technology relates to binding water-miscible solubilizers to drugs by secondary interactions, such as intermolecular or intramolecular hydrogen bonding, hydrophobic interaction, and the like to control the solubility of such drugs in aqueous environments. For example, the chemical conjugate of heparin and deoxycholic acid (heparin-DOCA) was developed for the oral delivery of heparin. U.S. Pat. No. 6,245,753; U.S. Pat. No. 6,656,922. However, heparin-DOCA is not effectively absorbed in the intestine because it cannot be dissolved in the gastrointestinal (GI) tract. On the other hand, when dimethylsulfoxide (DMSO) molecules, for example, are bound to heparin-DOCA, the solubility of heparin-DOCA in aqueous solution is greatly increased, thereby significantly enhancing its absorption in the GI tract. Therefore, the present invention significantly increases drug solubility and absorption in the intestine because of the effects of the bound solubilizer molecules. In particular, since the present technology uses a very small amount of solubilizer, the final product can be prepared as in powder form, which can be formulated as tablet or capsule type. Also, the toxicity of the solubilizer is negligible du to the small amount necessary to achieve the effect.

Since heparin cannot penetrate the intestinal bilayer because of its low partition coefficient and diffusivity in tissues, heparin-DOCA was synthesized by conjugation of heparin and deoxycholic acid for oral delivery. Heparin-DOCA was expected to interact readily with intestinal membranes, as well have a high partition coefficient in tissues. However, heparin-DOCA did not exhibit high absorption in the intestine due to its amphiphilicity. Amphiphilic heparin-DOCA made self-assembled particles under aqueous conditions, because of hydrophobic interactions between the conjugated DOCA molecules.

By the binding of organic molecules (dimethyl sulfoxide or N-methyl pyrrolidone, for example) to protein or polysaccharide, it is possible to change the molecular conformation of these molecules, which actually was changed quite freely in aqueous condition. Organic molecules intercalated in protein or polysaccharide affect the formation of aggregates formed by hydrophobic or ionic interactions. Therefore, randomly intercalating organic molecule in proteins or polysaccharides can block the formation of molecular aggregation or the interactions between molecules, thereby solubilizing such molecules in aqueous solution. For example, because of intercalating DMSO molecules, DMSO-bound heparin-DOCA conjugate do not self-aggregate in aqueous solution, thereby completely dissolving in aqueous media.

Dimethyl sulfoxide (CAS No. 67-68-5, DMSO, (CH₃)₂SO) is an important industrial solvent as well as being widely used in biology as a cryoprotector in the denaturation of proteins and as a drug carrier across cell membranes. Merck Index, Monograph No. 3285, 573 (13^{th} ed. 2001); D. Martin & H. G. Hauthal, Dimethyl Suphoxide (Wiley, New York 1975). Its broad range of properties is closely related to its properties in water solutions. DMSO mixes with water at all proportions. The partial negative charge on the oxygen atom of the DMSO molecule favors the formation of hydrogen bonds with water molecules, giving rise to strongly nonideal behavior of the mixture. Molecular dynamics results show that DMSO typically forms two hydrogen bonds with water molecules, although other configurations may also form. Hydrogen bonds between DMSO and water molecules are longer lived than water-water hydrogen bonds. A. Luzar & D. Chandler, Structure and hydrogen bond dynamics of water-dimethyl sulfoxide mixtures by computer simulations, 98 J. Chem. Phys. 8160-8173 (1993). The interaction between DMSO and water molecules is stronger than the interaction between water and the polar head group of a phospholipid membrane. S. N. Shashkov et al., The study of DMSO/water and DPPC/DMSO/water system by means of X-ray, neutron small angle scattering, calorimetry, and IR spectroscopy, 271 Physical B 184-191 (1999).

NMP (CAS No. 872-50-4) is a dipolar aprotic solvent, which is commercially prepared by condensation of butryolactone with methylamine. It is an industrial solvent used primarily in the extraction of aromatics from lubrication oils, but also has use in other applications, such as recovery and purification of acetylenes, olefins and diolefins, gas purification, aromatics extraction from feedstocks, and a polymer solvent. NMP is miscible with water, as well as with alcohol, ether, acetone, ethyl acetate, chloroform, and benzene. Merck Index, Monograph no. 6140, 1090 (13^{th} ed. 2001).

Diethylene glycol monoethyl ether (CAS No. 111-90-0), also known as 1-(2-ethoxyethoxy)ethanol and carbitol®, is a water-miscible solvent, which is prepared from ethylene oxide and 2-ethoxy-ethanol in the presence of SO₂. It is also miscible with acetone, benzene, chloroform, ethanol, ether, pyridine, and the like. It is used as a solvent for cellulose esters and in lacquer and thinner formulations, in quick-drying varnishes and enamels, and for dye-stuffs and wood stains. Merck Index, Monograph No. 1809, 302 (13^{th} ed. 2001).

Benzoic acid (CAS No. 65-85-0) is readily soluble in water, as well as in a variety of other solvents, such as alcohol, carbon tetrachloride, chloroform, ether, acetone, benzene, carbon disulfide, and the like. Benzoic acid is used for preserving foods, fats, fruit juices, and alkaloidal solutions; in the manufacture of benzoates, benzoyl compounds, and dyes; as a mordant in calico printing; for curing tobacco; and as an antifungal. Merck Index, Monograph 1092, 187 (13^{th} ed. 2001).

Heparin is a polysaccharide composed of sulfated D-glucosamine and D-glucuronic acid residues. Due to its numerous ionizable sulfate groups, heparin possesses a strong electronegative charge. It is also a relatively strong acid that readily forms water-soluble salts, e.g. heparin sodium. It is found in mast cells and can be extracted from many body organs, particularly those with abundant mast cells. The liver and lungs are especially rich in heparin. The circulating blood contains no heparin except after profound disruption of mast cells. Heparin has many physiological roles, such as blood anticoagulation, inhibition of smooth muscle cell proliferation, and so forth. In particular, heparin is a potent anticoagulant agent that interacts strongly with antithrombin m to prevent the formation of fibrin clots. In vivo, however, applications of heparin are very limited. Because of its hydrophilicity and high negative charge, heparin is not absorbed efficiently from the GI tract, nasal or buccal mucosal layers, and the like. Therefore, the only routes of administration used clinically are intravenous and subcutaneous injections.

U.S. Pat. No. 6,245,753 and U.S. Pat. No. 6,656,922 disclose amphiphilic heparin derivatives, that is, hydrophobized heparin conjugates wherein bile acids, sterols, alkanoic acids, and the like are conjugated to heparin for increasing the hydrophobicity of the very hydrophilic heparin molecule. Increasing the hydrophobicity of heparin by bonding a hydrophobic agent thereto results in what may be termed an amphiphilic heparin derivative or a hydrophobic heparin derivative. Either term is proper because the heparin derivative has increased hydrophobicity as compared to native heparin, and the heparin derivative

### EXAMPLE 1

A heparin-deoxycholic acid conjugate (heparin-DOCA) was prepared according to the method described in U.S. Pat. No. 6,656,922. Briefly, DOCA was activated by reaction with N-hydroxylsuccinimide (HOSu) and dicyclohexylcarbodiimide (DCC). Activated DOCA was then conjugated with heparin, and the resulting heparin-DOCA conjugate was purified by reverse phase and phenyl-Sepharose chromatography. Finally, purified heparin-DOCA was freeze dried at −80° C. to result in a white powder.

Next, aliquots of heparin-DOCA conjugate were dissolved in 10%, 30%, 50%, 70% and 90% aqueous DMSO solution, respectively. Briefly, heparin-DOCA (100 mg) was dissolved in five aliquots each of 500 μl of distilled water. To each aliquot 1, 3, 5, 7, or 9 ml of DMSO was added. The resulting solutions were dispersed by sonication with a probe-type sonicator. Finally, distilled water was added to each solution to make a final volume of 10 ml, and these solutions were well mixed before freeze drying at −80° C. for 3 days to obtain a white powder. The resulting preparations contained DMSO molecules and DMSO/water complexes bonded to the hydroxyl groups of the heparin-DOCA conjugate, as well as simple mixtures of DMSO, DMSO/water complexes, and heparin.

### EXAMPLE 2

The procedure of Example 1 was repeated except that N-methyl pyrrolidone (NMP) was substituted for DMSO.

### EXAMPLE 3

The procedure of Example 1 was repeated except that polyoxyl 35 castor oil (CREMOPHOR® EL; BASF) was substituted for DMSO.

### EXAMPLE 4

The procedure of Example 1 was repeated except that diethylene glycol monoethyl ether was substituted for DMSO.

### EXAMPLE 5

The procedure of Example 1 was repeated except that benzoic acid was substituted for DMSO.

### EXAMPLE 6

To determine how many DMSO molecules were contained in heparin-DOCA samples prepared according to the procedure of Example 1, samples containing 30 mg of heparin-DOCA were weighed before and after being freeze dried. DMSO-bound heparins and DMSO-bound heparin-DOCA conjugates were prepared with various amount of DMSO molecules. As shown in Table 1, increasing the DMSO concentration resulted in increased amounts of DMSO molecules bound to heparin-DOCA. When a 50% or higher DMSO solution was used, the amount of DMSO bound to heparin-DOCA was saturated.

TABLE 1

| | % DMSO | Final weight (mg) | DMSO Content (%) |
|---|---|---|---|
| 30 mg heparin- | 10 | 35 | 14.3 |
| DOCA | 30 | 45 | 33 |
| | 50 | 50 | 40 |
| | 70 | 40 | 25 |
| | 90 | 40 | 25 |
| 30 mg heparin | 70 | 43.5 | 31 |
| 50 mg heparin | 70 | 71.2 | 30.3 |
| 100 mg heparin | 70 | 145 | 31.5 |
| 100 mg DOCA | 70 | 123 | 18.7 |

### EXAMPLE 7

The procedure of Example 6 was repeated except that N-methylpyrrolidone, polyoxyl 35 castor oil, diethyleneglycol monoethylether, and benzoic acid were substituted for DMSO. The weight of the heparin-DOCA conjugates increased in these solvents, however, freeze drying showed that these compositions did not make self-assembled aggregates in water.

### EXAMPLE 8

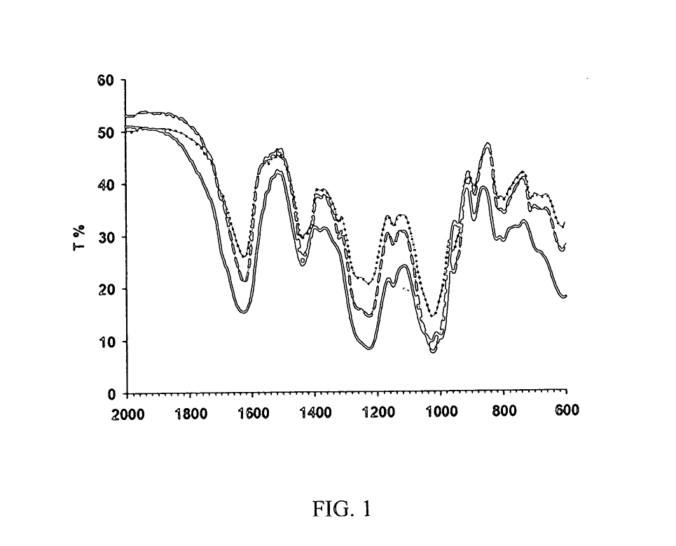

Fourier Transform Infrared spectroscopy (FT-IR) analysis was carried out according to methods well known in the art on heparin-DOCA samples mixed with DMSO. FT-IR data confirmed that DMSO molecules incorporated in heparin-DOCA were merely mixed or tightly bound with heparin-DOCA conjugates. Stretch or shift bands showed by the bonds of DMSO were observed from the results of FT-IR, as shown in FIG. 1. The shift of OH—bonds in DMSO-bound heparin-DOCA was shown at 1650 cm⁻¹. The stretching C—H vibrations of heparin-DOCA was located in the region 1317 and 1420 cm⁻¹. Also, rocking C—H bonds were observed at 950 cm⁻¹. The strong S—O stretch band (ν(S—O), DMSO) was observed at 1012-1014 cm⁻¹. These results show that DMSO molecules were bound with heparin-DOCA conjugates as well as being merely mixed with such conjugates.

Example 9

To analyze the characterization of DMSO-bound heparin-DOCA, thermal gravimetry analysis (TGA) and differential scanning calorimetry (DSC) were performed on heparin, DMSO-bound heparin, heparin-DOCA, and DMSO-bound heparin-DOCA.

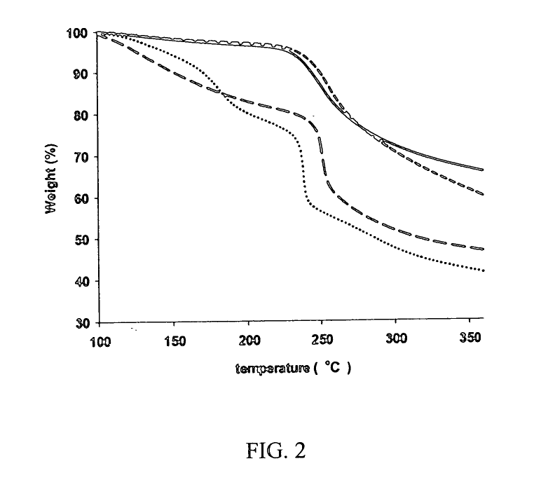

TGA determines the mass change of a sample as a function of temperature or time. FIG. 2 shows the results of the TGA experiment, wherein the total weight of DMSO-bound heparin-DOCA was changed at increasing temperatures by the evaporation of DMSO.

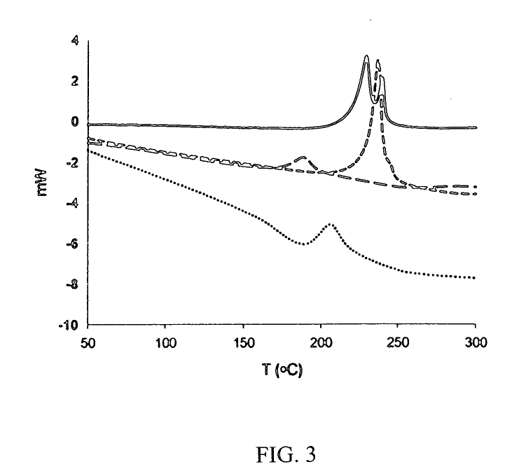

DSC is a technique for measuring the energy necessary to establish a nearly zero temperature difference between a substance and an inert reference material, as the two substances are subjected to identical temperature regimes in an environment heated or cooled at a controlled rate. FIG. 3 shows the results of the DSC experiment, wherein peaks present for heparin-DOCA disappeared when DMSO was present.

These results show that DMSO molecules may be bound or simply mixed with macromolecules. The binding of DMSO molecules to macromolecules may change the conformation of the macromolecules from a more ordered form to an amorphous form. DMSO molecules incorporated into the heparin-DOCA conjugates appears to affect the aggregate formation of amphiphilic heparin-DOCA and may reduce the crystallinity of heparin-DOCA. According to the increase of temperature, the total weight of DMSO-bound heparin-DOCA can be changed by the evaporation of DMSO molecule. Also, DSC profile will be differed by the change of the heparin-DOCA structure intercalating of DMSO molecules. From the DSC and TGA results, we found that DMSO molecules are bound or mixed with macromolecules. Therefore, by the binding of DMSO molecules, the structure of heparin-DOCA might be changed to the amorphous form. From the below figures, we observed that specific peaks of heparin-DOCA gradually disappeared by the incorporation of DMSO molecules

### EXAMPLE 10

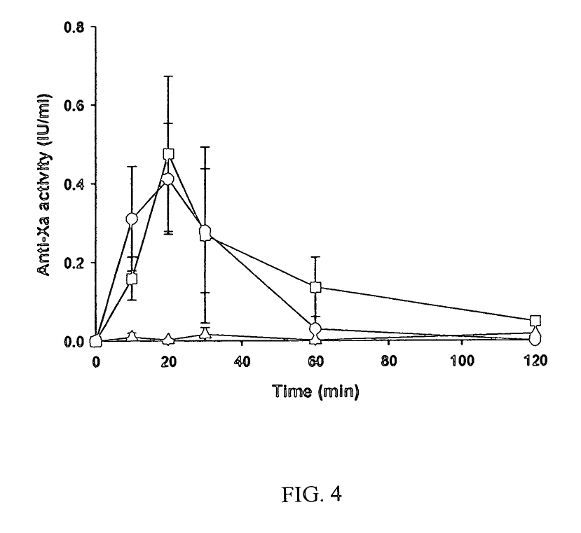

DMSO-bound heparin-DOCA was dissolved in distilled water, and it was determined that particles of DMSO-bound heparin-DOCA were not formed under these conditions. Accordingly, aqueous solutions of DMSO-bound heparin-DOCA were orally administered to mice (5 mg/kg) FIG. 4 shows that the absorption of DMSO-bound heparin-DOCA was significantly increased as compared to the absorption of DMSO-bound heparin. Further the bioavailability of DMSO-bound heparin-DOCA was not reduced as compared to heparin-DOCA in 10% aqueous DMSO.

### EXAMPLE 11

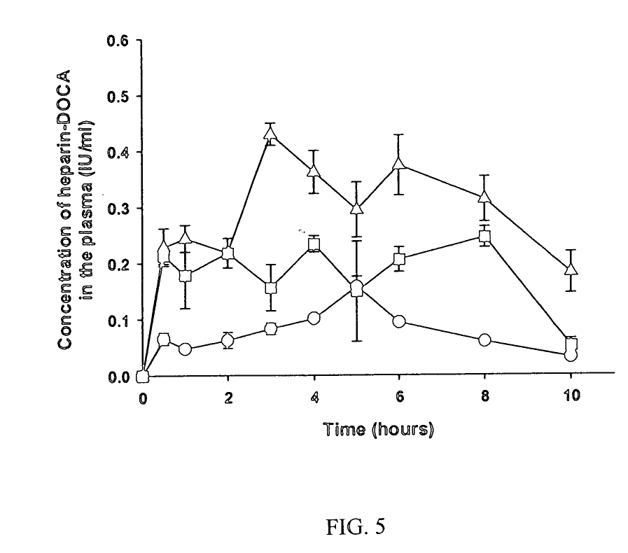

DMSO-bound heparin-DOCA prepared according to the method of Example 1 was encapsulated and then administered orally to monkeys at either 5 mg/kg or 10 mg/kg dosages. As a control, heparin in buffer was orally administered at 100 mg/kg. FIG. 5 shows that absorption of heparin was negligible, however, absorption of DMSO-bound heparin-DOCA was substantial and increased according to dosage.

### EXAMPLE 12

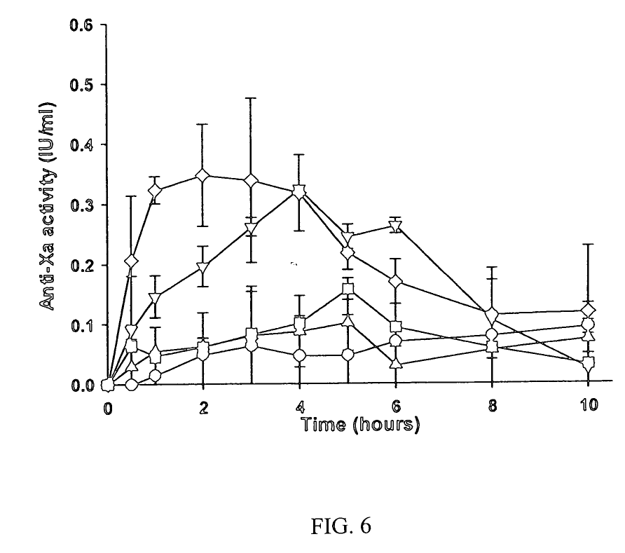

It was determined that DMSO-bound heparin-DOCA does not form particles, while heparin-DOCA does form particles, when dissolved or dispersed in water. In this example, aqueous formulations of heparin, heparin-DOCA, DMSO-bound heparin, and DMSO-bound heparin-DOCA were orally administered to monkeys. FIG. 6 shows that DMSO-bound heparin-DOCA at 5 mg/kg and 10 mg/kg dosages resulted in better absorption than any of the controls, namely, heparin (100 mg/kg), heparin-DOCA (10 mg/kg), and DMSO-bound heparin (10 mg/kg). Therefore, aqueous formulations of DMSO-bound heparin-DOCA are absorbed after oral administration, just as encapsulated powders are absorbed after oral administration (e.g., Example 11).

### EXAMPLE 13

Protein-bile acid conjugates were chemically synthesized. Namely, insulin-deoxycholic acid, insulin-lithocholic acid, insulin-cholic acid, calcitonin-deoxycholic acid, calcitonin-bis deoxycholic acid, and calcitonin-tris deoxycholic acid were synthesized according to methods well known in the art. These protein-bile acid conjugates were separately mixed with DMSO, polyoxyl 35 castor oil, N-methylpyrrolidone, diethyleneglycol monoethylether, and benzoic acid at 10%, 30%, 50%, 70%, and 90% organic solvent concentrations and then were freeze dried at −80° C. for 3 days.

The subject matter claimed is:

1. A composition comprising an aqueous mixture, complex, or combination of a mixture and a complex of a drug and a water-miscible organic solvent.

2. The composition of claim 1 wherein the drug is a member selected from the group consisting of polysaccharides, proteins, polysaccharide conjugates, protein conjugates, and protein complexes, and mixtures thereof.

3. The composition of claim 1 wherein the drug comprises a polysaccharide.

3. The composition of claim 1 wherein the drug comprises heparin.

4. The composition of claim 1 wherein the drug comprises a protein.

5. The composition of claim 1 wherein the drug comprises insulin.

6. The composition of claim 1 wherein the drug comprises calcitonin.

7. The composition of claim 1 wherein the drug comprises a conjugate of a polysaccharide and a bile acid, sterol, or alkanoic acid.

8. The composition of claim 1 wherein the drug comprises a conjugate of heparin and a bile acid.

9. The composition of claim 8 wherein the bile acid comprises deoxycholic acid.

10. The composition of claim 1 wherein the drug comprises a conjugate of a protein and a bile acid.

11. The composition of claim 10 wherein the drug comprises an insulin-bile acid conjugate.

12. The composition of claim 10 wherein the drug comprises a calcitonin-bile acid conjugate.

13. The composition of claim 1 wherein the water-miscible organic solvent is a member selected from the group consisting of dimethyl sulfoxide, N-methylpyrrolidone, polyoxyl 35 castor oil, diethylene glycol monoethyl ether, benzoic acid, and mixtures thereof.

14. The composition of claim 1 wherein the water-miscible organic solvent comprises dimethyl sulfoxide.

15. A composition formed by a process comprising:
(a) mixing a drug and a water-miscible organic solvent in an aqueous solution to form a mixture; and
(b) evaporating the mixture to dryness.

16. The composition of claim 15 wherein the drug comprises heparin and the organic solvent comprises dimethyl sulfoxide.

17. A method of treating a condition in a warm-blooded animal in need of treatment therefor, the method comprising orally administered a composition comprising a mixture, a complex, or a combination of a mixture and a complex of a drug and a water-miscible organic solvent.

18. The method of claim 17 wherein the composition comprises an aqueous solution.

19. The method of claim 17 wherein the composition comprises a dried form.

20. A composition comprising a mixture, a complex, or a combination of a mixture and a complex of heparin and dimethyl sulfoxide.

21. The composition of claim 20 wherein the composition comprises an aqueous solution.

22. The composition of claim 20 wherein the composition comprises a dried form.

23. A method of treating a patient in need of anticoagulation therapy, the method comprising orally administering a safe and effective amount of a composition comprising a mixture, a complex, or a combination of a mixture and a complex of a heparin-bile acid conjugate and dimethyl sulfoxide.

24. The method of claim 23 wherein the heparin-bile acid conjugate comprises heparin-deoxycholic acid complex.

25. A method of making an oral formulation for anticoagulation therapy, the method comprising:
(a) conjugating heparin to a bile acid to form a heparin-bile acid conjugate; and
(b) mixing the heparin-bile acid conjugate with an aqueous solution of a water-miscible organic solvent to form an aqueous formulation.

26. The method of claim 25 wherein the heparin-bile acid conjugate comprises heparin-deoxycholic acid conjugate.

27. The method of claim 25 wherein the water-miscible organic solvent comprises dimethyl sulfoxide.

28. The method of claim 25 further comprising evaporating the aqueous formulation to dryness.

29. The method of claim 28 further comprising encapsulating or tableting the dried aqueous formulation.

30. The method of claim 25 further comprising encapsulating the aqueous formulation.
